(19) United States
(12) Patent Application Publication
(10) Pub. No.: US 2004/0254424 A1
(43) Pub. Date: Dec. 16, 2004

(54) Integrated panoramic and forward view endoscope

(75) Inventors: Michelle D. Simkulet, Cohoes, NY (US); Jason E. Smith, Latham, NY (US); Ronald Gamache, East Greenbush, NY (US); Jiayin Ma, Rensselaer, NY (US)

Correspondence Address: Michelle Simkulet, 105 Jordan Road, Troy, NY, 12180, US

(73) Assignee: InterScience, Inc.

(21) Appl. No.: 10/822,964

(22) Filed: Apr. 13, 2004

Related U.S. Application Data
(60) Provisional application No. 60/462,951, filed on Apr. 15, 2003.

(51) Int. Cl. A61B 1/06

(52) U.S. Cl. 600/176; 600/109

(57) ABSTRACT

The objective of the present invention is to provide a single endoscope that provides a field of view substantially greater than a hemisphere comprising a forward field of view and a panoramic field of view that are integrated on a single image plane. The invention is described with respect to a rigid endoscope, but the technology can be implemented on a flexible endoscope as well. The advantage of such an endoscope is that it would provide substantially more information to the physician than any single existing endoscope, and it can be used in place of multiple endoscopes with varying directions of view that are swapped throughout a procedure to provide different views. The invention can also be used in non-medical applications for inspection in closed or generally inaccessible spaces such as for example the interior of jet engines.

### CROSS-REFERENCE TO RELATED APPLICATION

This application claims the benefit of U.S. provisional application No. 60,462,951, filed Apr. 15, 2003.

### FIELD OF THE INVENTION

This invention relates to the field of endoscopic imaging, and particularly to the imaging and illumination design of an endoscope that integrates on a single image plane a forward field of view (FFOV) and a panoramic field of view (PFOV), thereby providing the user a total field of view comprising the FFOV and the PFOV simultaneously.

### BACKGROUND OF THE INVENTION

Current art in endoscope design typically provides a 35° total viewing angle that may be rotated elevationally by angles up to 70° using prisms and mirrors. Further the view may be rotated axially by means of sheaths containing additional prisms/mirrors. Wide-angle views up to 120° are known but such designs suffer from high distortion and difficult component fabrication due to the need for aspherical or highly curved elements. For many medical procedures, such as nasal sinoscopy, the restricted viewing provided by current art endoscopes require that several endoscopes or different viewing angles be used at different points in the procedure. The act of withdrawing and inserting the endoscope, especially since the process may be somewhat blind, can be the cause of additional trauma to the patient. Panoramic imaging systems are known in the art but either do not have forward viewing or accomplish forward viewing in a different way than the current invention. U.S. Pat. No. 6,028,719 assigned to InterScience, Inc. discloses a general technique of integrating a forward view and a panoramic view utilizing a single reflector for the panoramic field of view.

There are many existing patents for optical systems that provide omnidirectional imaging. We believe we have some unique characteristics that are not covered in any existing patent and that provide a unique new capability to imaging systems and omnidirectional optical components in general. Jeffrey Charles has several U.S. patents on the subject including U.S. Pat. No. 6,333,826 and U.S. Pat. No. 6,449,103, BeHere Corporation has several US patents including U.S. Pat. No. 6,392,687, U.S. Pat. No. 6,424,377 and U.S. Pat. No. 6,480,229, and Remote Reality has U.S. Pat. No. 6,611,282.

The patents by Jeffrey Charles focus solely on the panoramic field of view, and efforts to maximize that field of view for near field applications. The Charles' patents include a frontal exclusion zone of about 60 degrees that can be tapered approaching the far field by the use of a torroidal-shaped reflector. Although this exclusion zone eventually disappears as a point where the boundaries of the panoramic field meet, there is no account in the patent for the overlapping area past the point of convergence in the processing or interpretation of the image. The minor disclosure of including forward optics to image the frontal exclusion zone makes no mention of details of how to match the magnification or the relative F/# of the integrated images as well as a means of interpreting or processing the overlapping images. The mere inclusion of forward viewing lenses does not automatically lend itself to an easily interpretable image. The focus of the optical system is near field prior to the overlap. Although there is provision to include the forward viewing optics to image the frontal exclusion zone, there will only be one point (or one radial distance) in which the frontal zone and the panoramic zone exist with either no gap or no overlap.

The BeHere technology also concentrates on the panoramic field of view and only makes provisions to extend the panoramic view as far forward as possible by changing the shape of the reflector. By placing a dimple in the apex of the parabolic reflector, imaging beyond the secondary reflector is achieved in the far field. These inventions provide no means for forward imaging in the near field.

The Remote Reality invention is a super wide-angle panoramic imaging apparatus that claims up to a 260° vertical field of view using a two reflector configuration. The invention includes an undefined blind spot along the optical axis. The invention claims a single view point while also having a substantially flat and stigmatic image plane.

None of these omnidirectional viewing systems provide a means of incorporating the optical system in an endoscope or borescope.

### OBJECTS OF THE INVENTION

It is an object of the present invention to provide a means of integrating panoramic imaging capabilities with a forward viewing endoscope design.

It is an object of the present invention to provide a means of integrating panoramic imaging capabilities with a forward viewing endoscope design utilizing a two reflector panoramic imaging component.

It is an object of the present invention to provide an endoscope design capable of presenting a forward field of view and a panoramic field of view integrally on a single image plane.

It is an object of the present invention to provide a total field of view that is upright and unreversed without need for extensive computer processing to accomplish said upright and unreversed field of view.

It is an object of the present invention to provide an endoscope design in which the boundaries of the forward field of view and panoramic field of view can be customized to fit to specific application needs.

It is an object of the present invention to provide illumination means for a forward field of view and a panoramic field of view of an endoscope.

It is an object of the present invention to provide an endoscope capable of an integrated panoramic and forward view that can approach or exceed a solid angle of 2π steradians.

It is an object of the present invention to provide the total field of view with low distortion, chromatic aberration, and viewpoint error. Such qualities are necessary to support diagnostic assessments during intended medical procedures.

### SUMMARY OF THE INVENTION

The objective of the present invention is to provide a single endoscope that provides a total field of view substantially greater than a hemisphere comprising a forward field of view and a panoramic field of view that are integrated on a single image plane. The invention is described with respect to a rigid endoscope, but the technology can be implemented on a flexible endoscope as well. The advantage of such an endoscope is that it would provide substantially more information to the physician than any single existing endoscope, and it can be used in place of multiple endoscopes with varying directions of view that are swapped throughout a procedure to provide different views. The invention can also be used in non-medical applications for inspection in closed or generally inaccessible spaces, such as the interior of jet engines.

### BRIEF DESCRIPTION OF THE DRAWINGS

The present invention, and the objects and advantages thereof, may best be understood by reference to the following detailed description and accompanying drawings in which:

FIG. 1 is an overall view of the entire panoramic/forward view endoscope.

FIG. 2 is a longitudinal cross-section of the panoramic/forward view element and the endoscope objective.

FIG. 3 is an axial cross section of the distal tip of the panoramic/forward view endoscope.

FIG. 4 is an axial cross section of the relay and objective area of the panoramic/forward view endoscope.

FIG. 5 is a first embodiment of the illumination distribution.

FIG. 6 is a second embodiment of the illumination distribution.

FIG. 7 is a third embodiment of the illumination distribution.

FIG. 8 is a fourth embodiment of the illumination distribution.

FIG. 9 is a fifth embodiment of the illumination distribution.

FIG. 10 is a sixth embodiment of the illumination distribution.

FIG. 11 is a seventh embodiment of the illumination distribution.

### DETAILED DESCRIPTION

The present invention provides an endoscope design that provides a total field of view substantially greater than a hemisphere comprising a forward field of view and a panoramic field of view that are continuous and integrated on a single image plane. The integrated fields of view are matched in magnification and brightness and there is a relatively seamless boundary between them with no blindspots or overlapping of the fields. The invention comprises panoramic and forward view imaging technology as well as panoramic and forward illumination technology. The invention is demonstrated on a rigid endoscope but the technology can be implemented on a flexible endoscope as well.

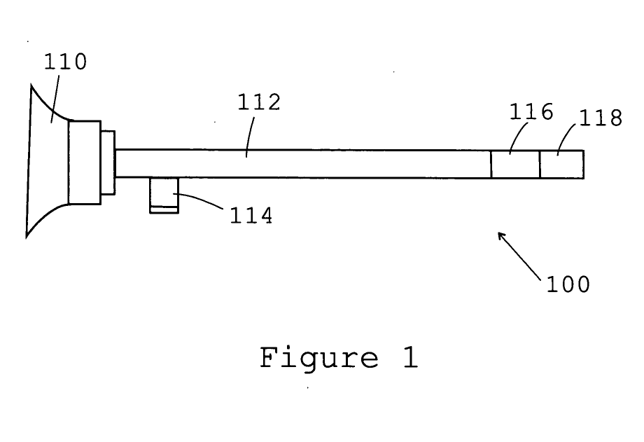

The present invention is initially described with respect to FIG. 1. FIG. 1 shows the panoramic/forward view endoscope 100; which comprises a rigid endoscope eyepiece 110, housing of a rigid endoscope relay system 112, an illumination light guide port 114, housing of a endoscope objective 116, and housing of an integrated panoramic/forward viewing optical element 118.

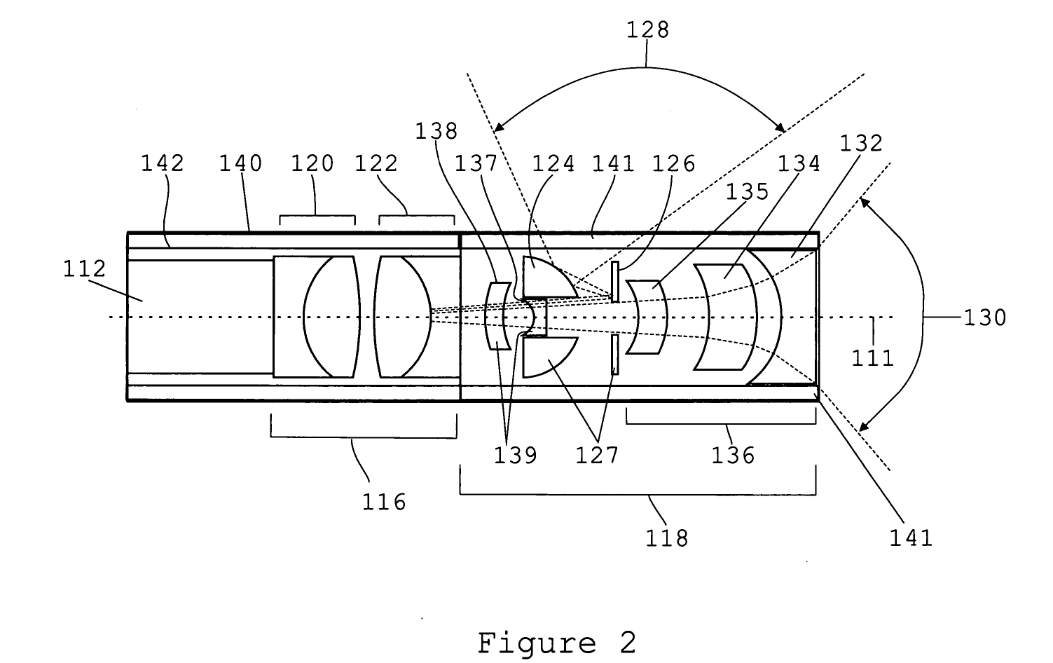

The present invention utilizes a endoscope eyepiece 110, an endoscope relay system 112, and an illumination light guide port 114 as known in the art. The improvements of the present invention to existing endoscope design are substantially provided in the endoscope objective 116 and the panoramic/forward viewing optical element 118. It is these elements that contribute to the unique 2π+ steradian (solid angle) viewing capabilities of the present invention. The layout of the modified endoscope objective 116 and the panoramic/forward viewing optical element 118 is shown in detail in FIG. 2.

As shown in FIG. 2, the endoscope objective 116 is adjacent to the endoscope relay system 112. The endoscope objective 116 essentially comprises at least one focusing element. The figure depicts an embodiment comprising a first focusing element 120 and a second focusing element 122. The endoscope objective 116 serves to transform the converging ray bundles collected by the panoramic/forward view element 118 into telecentric input for the endoscope relay system 112.

As shown in FIG. 2, the endoscope objective 116 is adjacent to and receives optical input from the panoramic/forward viewing optical element 118. The panoramic/forward viewing optical element 118 essentially comprises a Panoramic Field of View (PFOV) optical element group 127, a Forward Field of View (FFOV) optical element group 136, and a focusing optical element group 139. The PFOV optical element group 127 essentially comprises two reflectors each having one mirror surface and each having a central aperture. A first reflector 124 is essentially a solid convex surface with the mirrored surface facing the distal end of the endoscope 100 and a central aperture. The first reflector 124 is symmetric about its central axis and central aperture and is aligned along the optical axis 111. A cross-section of the first reflector 124, as depicted in FIG. 2, would show the reflective surface to be a portion of a mathematical conic section, such as but not limited to a sphere or a parabola. A second reflector 126 with mirror surface facing the first reflector 124 can be planar, concave or convex. The surface geometry of both the first reflector 124 and the second reflector 126 can be optimized to obtain the desired PFOV 128 for a specific application.

The Forward Field of View (FFOV) optical element group 136 is comprised of a first lens group 132, a second lens group 134, and a third lens group 135 that images portions of the object substantially distal to the endoscope, i.e. the FFOV 130. The first lens group 132 gathers rays from a wide angle centered on the optical axis 111. The second and third lens groups 134, 135 focus and reduce the size of the gathered ray bundle so that it may pass through the apertures of the first and second reflectors 124 and 126.

The focusing optical element group 139 is centered along the optical axis 111 and is placed in line in the optical path between the PFOV optical element group 127 and the endoscope objective 116. It comprises at least two focusing optical elements, a first focusing optical element 137 and a second focusing optical element 138. The focusing optical element group 139 collects the panoramic field of view 128 from the secondary reflector 126 and the forward field of view 130 from the FFOV optical element group 136. It is the function of the focusing optical element group 139 to focus the two independent optical paths from the panoramic field of view 128 and the forward field of view 130 as a coplanar image and to control the image aberrations on this coplanar image.

As shown in FIG. 2, image information from the PFOV is collected by the first reflector 124 and is then reflected onto the second reflector 126. The second reflector 126 then reflects the image information through the central aperture of the first reflector 124 to the focusing optical element group 139 and the endoscope objective 116. The forward field of view optical element group 136 passes the image information of the forward field of view 130 through the central aperture of the second reflector 126 and the first reflector 124 to the focusing optical element group 139 and the endoscope objective 116. The geometries of the first and second reflectors 124 and 126 are designed to accept rays from the PFOV 128 and converge them with the FFOV 130 for coplanar focusing by the focusing optical element group 139 and the endoscope objective 116. The image information from the FFOV 130 and the PFOV 128 provide an overall field of view of approximately 240 degrees. The image information from the FFOV 130 and the PFOV 128 are matched substantially seamlessly on the image plane with virtually no overlap and no gap between them. The magnification and relative F# (or brightness) of the FFOV 130 and the PFOV 128 are matched as well.

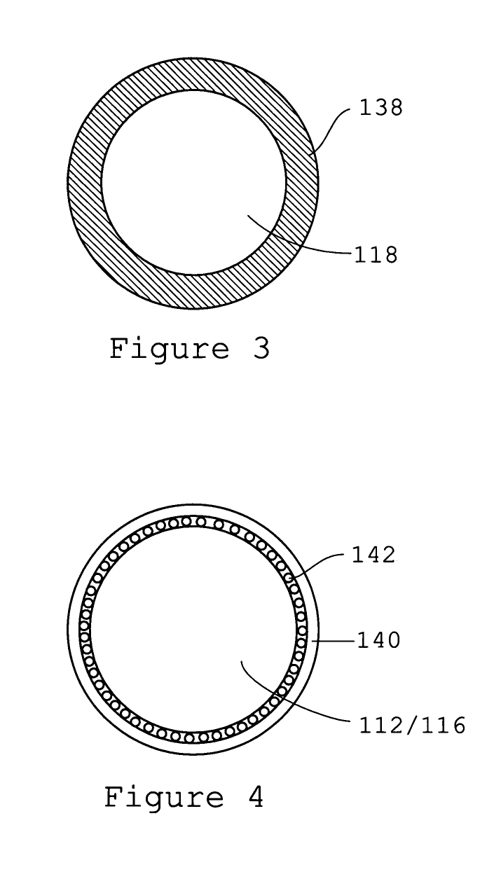

As shown in FIGS. 2 and 3, disposed circumferentially about a substantial portion of the panoramic/forward viewing optical element 118 is a transparent cylindrical tube 141 that provides structural support and sealing for the system as well as a means for rays from the PFOV 128 to enter the system. It is known in the art that panoramic imaging systems comprised of spherical reflectors suffer from so-called non-single viewpoint. Images from such non-single viewpoint systems cannot be processed to produce geometrically correct perspective views. For spherical reflector systems, each object point is viewed from a different viewpoint. Such variability of the viewpoint causes uncorrectable parallax in perspective views generated from such imagery. A further advantage of the transparent cylindrical tube 141 is to significantly reduce the size of the so-called viewpoint caustic and therefore parallax errors in the acquired perspective views. The viewpoint error can be brought to a minimum through the specification of the refractive index and thickness of the cylindrical tube 141.

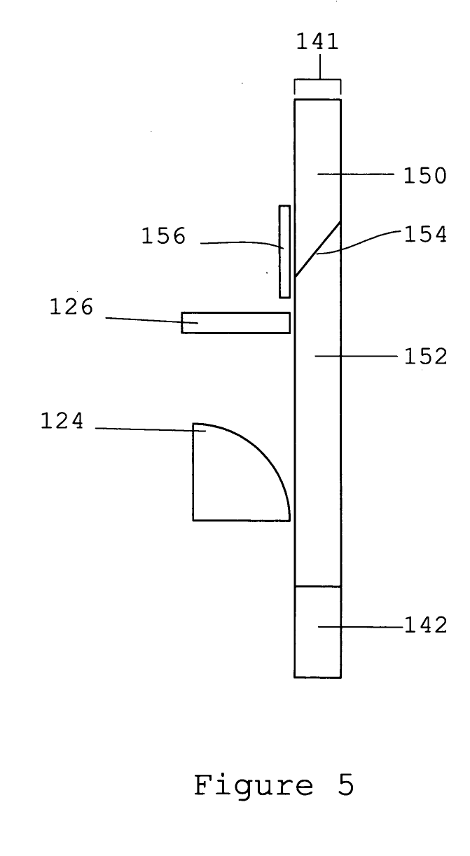

Shown in FIGS. 2 and 4, as the panoramic/forward viewing element 118 is encircled by the transparent cylindrical tube 147, the endoscope relay 112 and modified endoscope objective 116 are circumferentially encased by endoscope lumenal housing 140. The circumference of the endoscope lumenal housing 140 is lined by endoscope illumination means 142. This illumination is distributed to the PFOV 128 and the FFOV 130. FIGS. 5, 6, 7, 8, 9, 10, and 11 show several options for distributing the illumination to the PFOV 128 and the FFOV 130.

Shown in FIG. 5 is a first embodiment of the illumination distribution in the panoramic/forward view endoscope 100. In this embodiment the transparent cylindrical tube 141 comprises at least two sections, a distal section 150 and a proximal section 152 joined by an angled seam 154. In this embodiment a semi-transparent/semi-reflective coating could be introduced on the seam 154 so as to promote the proper distribution of the illumination between the periphery of the endoscope 100 and the distal end of the endoscope 100. An adequate interface is established between the endoscope illumination means 142 and the transparent cylindrical tube 141, such as but not limited to optically transparent adhesive. This embodiment could benefit from the optional addition of a rigid and opaque internal support 156 for added structural support and as a means of preventing internal light leakage.

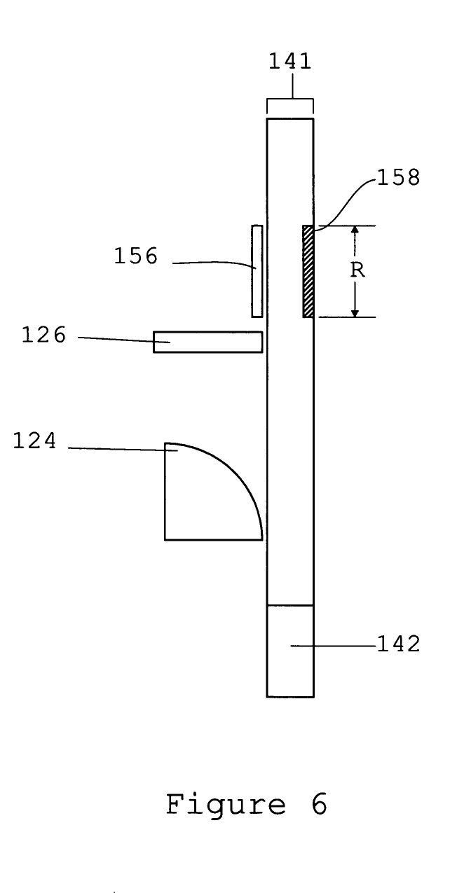

Shown in FIG. 6 is a second embodiment of the illumination distribution in the panoramic/forward view endoscope 100. In this embodiment, a diffuse ring 158 of width R is on the outer circumference of the solid transparent cylindrical tube 141. The diffuse ring 158 is located distal to the PFOV 128 so as not to interfere with the imaging in the PFOV 128. In this embodiment an adequate interface is established between the endoscope illumination means 142 and the transparent cylindrical tube 141, such as but not limited to optically transparent adhesive. This embodiment could benefit from the optional addition of a rigid and opaque internal support 156 for added structural support and as a means of preventing internal light leakage.

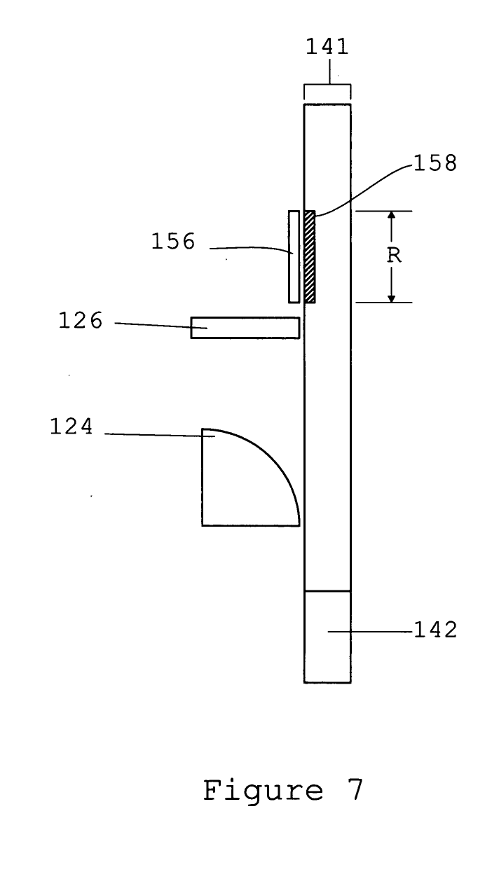

Shown in FIG. 7 is a third embodiment of the illumination means. In this embodiment, a diffuse ring 158 of width R is on the inner circumference of the solid transparent cylindrical tube 141. The diffuse ring 158 is located distal to the PFOV 128 so as not to interfere with the imaging in the PFOV 128. The diffuse ring 158 would radially scatter some of the light to illuminate the PFOV 128 that is propagating through the tube 141 to illuminate the FFOV 130. As in the first embodiment an adequate interface is established between the endoscope illumination means 142 and the transparent cylindrical tube 141, such as but not limited to optically transparent adhesive. This embodiment could benefit from the optional addition of a rigid and opaque internal support 156 for added structural support and as a means of preventing internal light leakage.

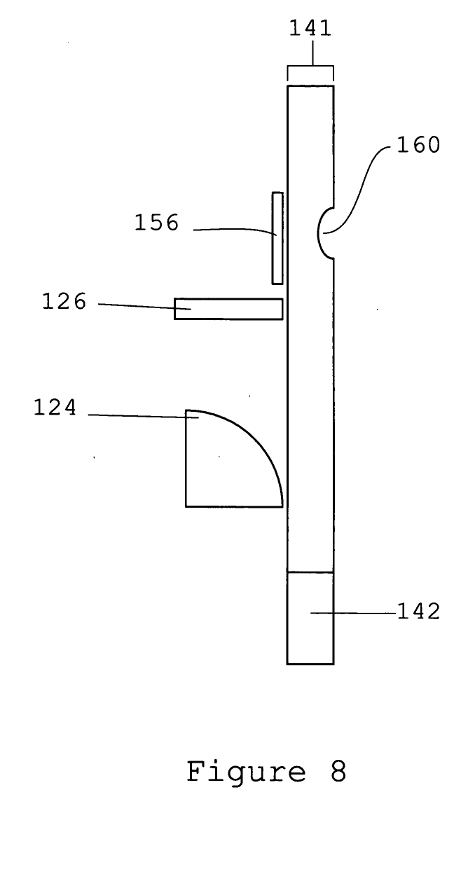

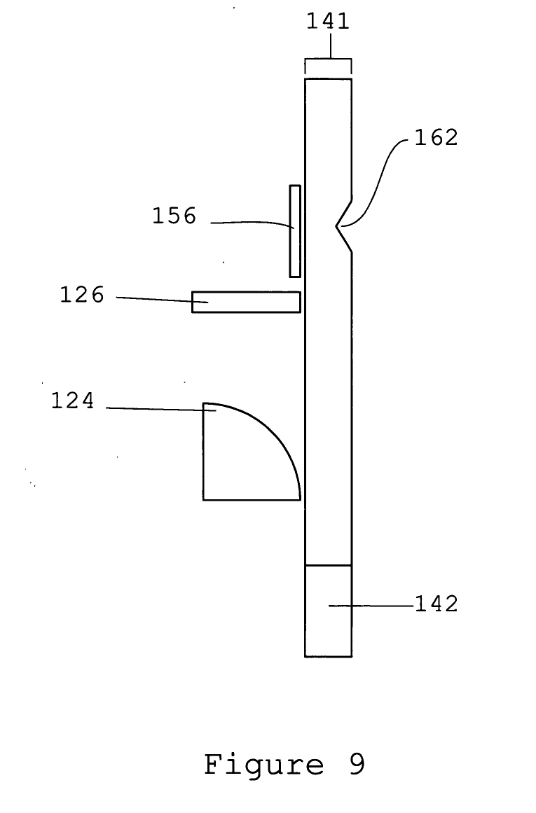

Shown in FIG. 8 is a fourth embodiment of the illumination distribution in the panoramic/forward view endoscope 100. In this embodiment, a curved notch 160 is on the outer circumference of the solid transparent cylindrical tube 141. The curved notch 160 is located distal to the PFOV 128 so as not to interfere with the imaging in the PFOV 128. The notch 160 is included to interrupt and divert the transmission of a portion of the illumination along the transparent cylindrical tube 141 and therefore allowing illumination to be distributed to the PFOV 128. As in the first embodiment an adequate interface is established between the endoscope illumination means 142 and the transparent cylindrical tube 141, such as but not limited to optically transparent adhesive. This embodiment could benefit from the optional addition of a rigid and opaque internal support 156 for added structural support and as a means of preventing internal light leakage. Alternatively the notch may be an angled notch 162 as shown in the fifth embodiment in FIG. 9.

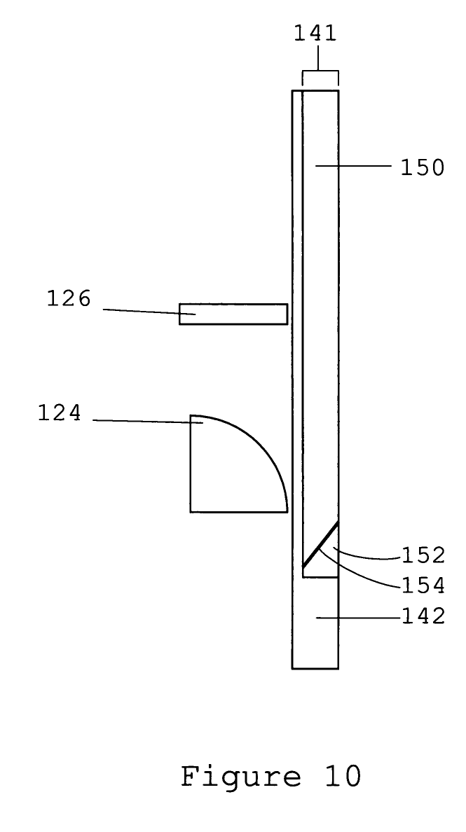

FIG. 10 shows a sixth alternative embodiment of the illumination means. In this embodiment a portion of the illumination fibers continue along the inner circumference of the transparent tube to illuminate the forward field of view. The remainder of the illumination fibers end at the proximal end of the transparent tube to distribute light to the panoramic field of view. The transparent cylindrical tube 141 comprises at least two sections, a distal section 150 and a proximal section 152 joined by an angled seam 154. In this embodiment a reflective coating is introduced on the seam 154 so as to promote the proper distribution of the illumination to the periphery of the endoscope 100. An adequate interface is established between the endoscope illumination means 142 and the transparent cylindrical tube 141, such as but not limited to optically transparent adhesive.

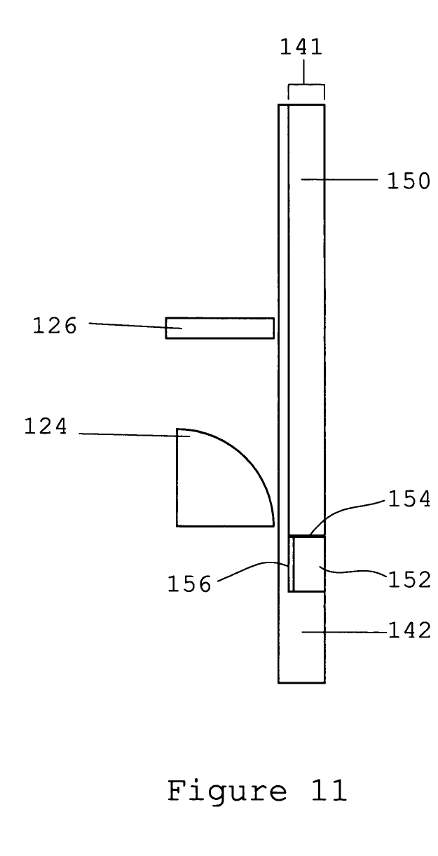

FIG. 11 shows a seventh alternative embodiment of the illumination means. In this embodiment a portion of the illumination fibers continue along the inner circumference of the transparent tube to illuminate the forward field of view. The remainder of the illumination fibers end at the proximal end of the transparent tube to distribute light to the panoramic field of view. The transparent cylindrical tube 141 comprises at least two sections, a distal section 150 and a proximal section 152 joined by a seam 154. In this embodiment a reflective coating is introduced on the seam 154 and the proximal section 152 is made entirely of diffuse glass with a light blocking barrier 156 on its inner diameter so as to promote the proper distribution of the illumination to the periphery of the endoscope 100. An adequate interface is established between the endoscope illumination means 142 and the transparent cylindrical tube 141, such as but not limited to optically transparent adhesive.

While only certain preferred features of the invention have been illustrated and described, many modifications, changes and substitutions will occur to those skilled in the art. It is, therefore, to be understood that this disclosure and its associated claims are intended to cover all such modifications and changes as fall within the true spirit of the invention

We claim:

1. An endoscopic optical system comprising;
a panoramic/forward viewing optical element which collects image information from the forward field of view and the panoramic field of view; and
an endoscope objective that collects and focuses the image information from the panoramic/forward viewing optical element; and
an endoscopic eyepiece to view the image information; and
an endoscopic relay system to transmit image information through the endoscope from the endoscope objective to the endoscopic eyepiece; and
a means of endoscopic illumination to distribute light to the forward field of view and the panoramic field of view.

2. An endoscopic optical system according to claim 1, wherein the panoramic/forward viewing optical element, further comprises a forward field of view optical element group, a panoramic field of view optical element group and a focusing optical element group.

3. An endoscopic optical system according to claim 2, wherein the forward field of view optical element group further comprises at least one optical element group.

4. An endoscopic optical system according to claim 2, wherein the panoramic field of view optical element group further comprises a first reflector and a second reflector.

5. An endoscopic optical system according to claim 2, wherein the focusing optical element group further comprises at least on optical element group.

6. An endoscopic optical system according to claim 4, wherein the first reflector has a spherical geometry.

7. An endoscopic optical system according to claim 4, wherein the first reflector has an aspherical geometry.

8. An endoscopic optical system according to claim 4, wherein the second reflector has a planar geometry.

9. An endoscopic optical system according to claim 4, wherein the second reflector has a concave geometry.

10. An endoscopic optical system according to claim 4, wherein the second reflector has a convex geometry.

11. An endoscopic optical system according to claim 4, wherein the first reflector has a central clear aperture to pass the image information through.

12. An endoscopic optical system according to claim 4, wherein the second reflector has a central clear aperture to pass the forward field of view image information through.

13. An endoscopic optical system according to claim 1, wherein the image information viewed through the endoscopic eyepiece comprises the forward field of view image information and the panoramic field of view image information on a single image plane.

14. An endoscopic optical system according to claim 13, wherein the image information viewed through the endoscopic eyepiece comprises a total field of view of at least 240 degrees.

15. An endoscopic optical system according to claim 13, wherein the image information viewed through the endoscopic eyepiece comprises a substantially seamless boundary between the forward field of view image information and the panoramic field of view image information.

16. An endoscopic optical system according to claim 13, wherein the image information viewed through the endoscopic eyepiece comprises substantially matched magnifications for the forward field of view image information and the panoramic field of view image information.

17. An endoscopic optical system according to claim 13, wherein the image information viewed through the endoscopic eyepiece comprises substantially matched brightness for the forward field of view image information and the panoramic field of view image information.

18. An endoscopic imaging system according to claim 1, wherein the panoramic/forward viewing optical element is housed within an optically transparent tube that is integrally aligned with the remainder of the endoscope housing.

19. An endoscopic imaging system according to claim 1, wherein the means of illumination comprises fiber optic illumination around the entire outer circumference and a semi-reflective and semi-transparent angled seam in an optically transparent tube placed distally to the fiber optic illumination to distribute the illumination to both the forward field of view and the panoramic field of view.

20. An endoscopic imaging system according to claim 1, wherein the means of illumination comprises fiber optic illumination around the entire outer circumference and an optically transparent tube with a diffuse portion on its outer circumference placed distally to the fiber optic illumination to distribute the illumination to both the forward field of view and the panoramic field of view.

21. An endoscopic imaging system according to claim 1, wherein the means of illumination comprises fiber optic illumination around the entire outer circumference and an optically transparent tube with a diffuse portion on its inner circumference placed distally to the fiber optic illumination to distribute the illumination to both the forward field of view and the panoramic field of view.

22. An endoscopic imaging system according to claim 1, wherein the means of illumination comprises fiber optic illumination around the entire outer circumference and an optically transparent tube with a curved notch on its outer circumference placed distally to the fiber optic illumination to distribute the illumination to both the forward field of view and the panoramic field of view.

23. An endoscopic imaging system according to claim 1, wherein the means of illumination comprises fiber optic illumination around the entire outer circumference and an optically transparent tube with a angled notch on its outer circumference placed distally to the fiber optic illumination to distribute the illumination to both the forward field of view and the panoramic field of view.

24. An endoscopic imaging system according to claim 1, wherein the means of illumination comprises fiber optic illumination around the entire outer circumference with some fibers continuing on the inside of the optically transparent tube for illumination of the forward field of view and the remainder of the fibers ending at the optically transparent tube for illumination distribution to the panoramic field of view.

25. An endoscopic imaging system according to claim 24, wherein the optically transparent tube further comprises a reflective angled seam for illumination distribution to the panoramic field of view.

26. An endoscopic imaging system according to claim 24, wherein the optically transparent tube further comprises a reflective seam and an optically diffuse proximal section for illumination distribution to the panoramic field of view.
